(19) United States
(12) Patent Application Publication
(10) Pub. No.: US 2019/0365342 A1
(43) Pub. Date: Dec. 5, 2019

(54) METHOD AND SYSTEM FOR DETECTING ABNORMAL HEART SOUNDS

(71) Applicant: Robert Bosch GmbH, Stuttgart (DE)

(72) Inventors: Shabnam Ghaffarzadegan, San Mateo, CA (US); Zhe Feng, Mountain View, CA (US); Ahmed Imtiaz Humayun, Dhaka (BD); Taufiq Hasan, Syosset, NY (US)

(21) Appl. No.: 16/033,296

(22) Filed: Jul. 12, 2018

Related U.S. Application Data
(60) Provisional application No. 62/680,404, filed on Jun. 4, 2018.

(51) Int. Cl. A61B 7/04

(52) U.S. Cl. CPC A61B 7/04; A61B 5/7264

(57) ABSTRACT

A method and system for detecting abnormal heart sounds in a phonocardiogram of a person are disclosed. At least one segmented cardiac cycle of the phonocardiogram is received at a processor. The processor decomposes the segmented cardiac cycle into a plurality of frequency sub-bands using a first convolutional neural network having, in particular a plurality of time-convolution layers (tConv). The kernel weights of each time-convolution layer are learned in a training process such that the time-convolution layers identify pathologically significant frequency sub-bands. The processor determines a probability that the segmented cardiac cycle contains an abnormal heart sound based on the plurality of frequency sub-band segments using at least one further neural network. In some embodiments, the time-convolution layers are configured to have a linear phase response (LP-tConv) or a zero phase response (ZP-tConv).

This application claims the benefit of priority of U.S. provisional application Ser. No. 62/680,404, filed on Jun. 4, 2018 the disclosure of which is herein incorporated by reference in its entirety.

### FIELD

The device and method disclosed in this document relates to detecting abnormal heart sounds and, more particularly, to automated detection of abnormal heart sounds using neural networks.

### BACKGROUND

Unless otherwise indicated herein, the materials described in this section are not prior art to the claims in this application and are not admitted to the prior art by inclusion in this section.

Cardiovascular diseases (CVDs) are responsible for about 17.7 million deaths every year, representing 31% of the global mortality. Cardiac auscultation is the most popular non-invasive and cost-effective procedure for the early diagnosis of various heart diseases. However, effective cardiac auscultation requires trained physicians, a resource which is limited especially in low-income countries of the world. Automated classification of the Phonocardiogram (PCG), i.e., the heart sound, have been extensively studied and researched in the past few decades. Analysis of the PCG can be broadly divided into two principal areas: (i) segmentation of the PCG signal, i.e., detection of the first and second heart sounds (S1 and S2), and (ii) classification of recordings as pathologic or physiologic.

### SUMMARY

A method of detecting abnormal heart sounds in a phonocardiogram of a person is disclosed. The method comprises: receiving, with a processor, a first segment of the phonocardiogram, the first segment comprising a time series of acoustic values from the phonocardiogram; decomposing, with the processor, the first segment into a plurality of frequency sub-band segments using a first convolutional neural network having a plurality of kernel weights that were learned in a training process of the first convolutional neural network, each frequency sub-band segment comprising a time series of acoustic values corresponding to a respective frequency sub-band of first segment; determining, with the processor, a probability that the first segment contains an abnormal heart sound based on the plurality of frequency sub-band segments using at least one neural network; and generating, with an output device, a perceptible output depending on the probability that first segment contains the abnormal heart sound.

A system for detecting abnormal heart sounds in a phonocardiogram of a person. The system includes: a stethoscope having at least one acoustic sensor configured to record the phonocardiogram of the person and a transceiver configured to transmit the phonocardiogram; and a portable electronic device having a processor, an output device, and a transceiver. The processor is configured to: operate the transceiver to receive the phonocardiogram from the stethoscope; segment the phonocardiogram into a plurality of segments, each segment comprising a time series of acoustic values corresponding to only one cardiac cycle from the phonocardiogram; for each segment in the plurality of segments: decompose the respective segment into a respective plurality of frequency sub-band segments using the first convolutional neural network, each frequency sub-band segment comprising a time series of acoustic values corresponding to a respective frequency sub-band of the respective segment; and determine a probability that the respective segment contains the abnormal heart sound based on the respective plurality of frequency sub-band segments using at least one neural network; and operate the output device to generate a perceptible output depending on the probabilities that each segment in the plurality of segments contains the abnormal heart sound.

### BRIEF DESCRIPTION OF THE DRAWINGS

The foregoing aspects and other features of the method and system for detecting abnormal heart sounds are explained in the following description, taken in connection with the accompanying drawings.

FIG. 1 shows an exemplary embodiment of a system for detecting abnormal heart sounds of a person.

FIG. 2A shows an exemplary embodiment of a stethoscope of the system of FIG. 1.

FIG. 2B shows an exemplary embodiment of a portable electronic device of the system of FIG. 1.

FIG. 3 shows an exemplary embodiment of a phonocardiogram classification model.

FIG. 4 illustrates how the time-convolution layers function as a Finite Impulse Response (FIR) filter bank.

FIG. 5 shows an embodiment of a time-convolution layer in which a forward-reverse convolution is incorporated, such that the time-convolution layer has zero phase response.

FIG. 6 shows decompositions of a segmented cardiac cycle with an exemplary Finite Impulse Response filter bank, an exemplary time-convolution layer, and an exemplary linear phase time-convolution layer, as well as magnitude and phase responses of the exemplary linear phase time-convolution layer.

FIG. 7 shows a logical flow diagram for a method operating the system of FIG. 1 to detect abnormal hearts sounds of a heart of a person.

### DETAILED DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present disclosure includes any alterations and modifications to the illustrated embodiments and includes further applications of the principles of the disclosure as would normally occur to one skilled in the art which this disclosure pertains.

### Abnormal Heart Sound Detecting System

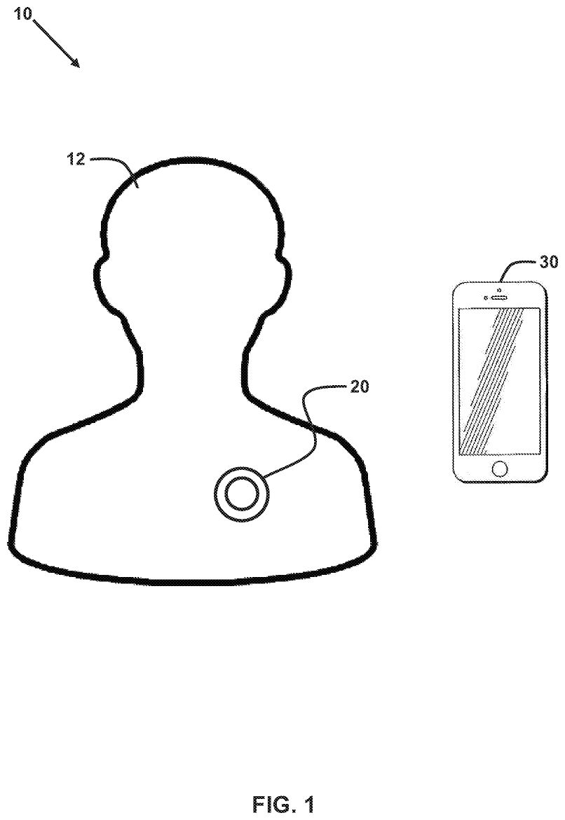

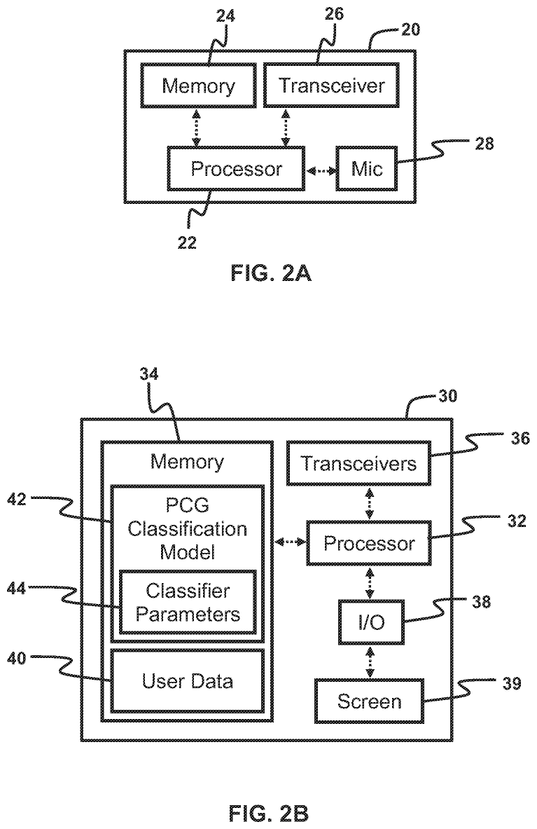

With reference to FIGS. 1 and 2A-2B, an exemplary embodiment of an abnormal heart sound detecting system 10 for detecting abnormal heart sounds of a person 12 is described. The abnormal heart sound detecting system 10 is configured to monitor acoustic characteristics of a heart sound of the person 12 and inform a user in case of abnormalities in heart. Particularly, as discussed in greater detail below, the abnormal heart sound detecting system 10 utilizes a machine learning model to classify heart sounds as normal or abnormal. In this way, the abnormal heart sound detecting system 10 can play a vital role in the early diagnosis of heart diseases, without the need for trained physicians.

As shown in FIG. 1, the abnormal heart sound detecting system 10 includes a stethoscope 20 and a portable electronic device 30. The stethoscope 20 is placed against the chest of the person 12 and is configured to record a sound recording of the heart of the person 12, referred to herein as a phonocardiogram (PCG). The stethoscope 20 digitizes and transmits the phonocardiogram to the portable electronic device 30. The portable electronic device 30 is configured to process the phonocardiogram to automatically detect abnormal heart sounds of the person 12. In the event that abnormal hearts sounds are detected, the portable electronic device 30 is configured to inform the user thereof (e.g., the person 12 himself or herself, a physician, a nurse, or other user) of whether the phonocardiogram includes any abnormal heart sounds.

FIG. 2A shows an exemplary embodiment of a stethoscope 20, which is configured to record a phonocardiogram of the heart of the person 12 and provide it to the portable electronic device 30. In the illustrated embodiment, the stethoscope 20 comprises a processor 22 operably connected with a memory 24, a transceiver 26, and a microphone 28. The memory 24 is configured to store program instructions that, when executed by the processor 22, enable the stethoscope 20 to perform various operations described elsewhere herein, including recording a phonocardiogram of the heart of the person 12 and communicating with the portable electronic device 30 to provide the phonocardiogram to the portable electronic device 30. The memory 24 may be of any type of device capable of storing information accessible by the processor 22, such as write-capable memories, read-only memories, or other computer-readable mediums. Additionally, it will be recognized by those of ordinary skill in the art that a “processor” includes any hardware system, hardware mechanism or hardware component that processes data, signals or other information. The processor 22 may include a system with a central processing unit, multiple processing units, dedicated circuitry for achieving functionality, or other systems.

The transceiver 26 at least comprises a transceiver, such as a Bluetooth® transceiver, configured to communicate with the portable electronic device 30, but may also include any of various other devices configured for communication with other electronic devices, including the ability to send communication signals and receive communication signals. It will be appreciated that, in alternative embodiments, the stethoscope 20 communicates with the portable electronic device 30 via a wired interface.

The microphone 28 comprises any type of acoustic sensor configured to record a phonocardiogram of the heart of the person 12 when placed near or against the chest of the person 12. Particularly, the microphone 28 is configured to convert sound waves and/or pressure changes corresponding to heart sounds into an electrical signal. In at least one embodiment, the stethoscope 20 and/or the microphone 28 includes an analog to digital converter (not shown) configured to convert an electrical signal corresponding to heart sounds into a digital phonocardiogram. In some embodiments, the microphone 28 may comprise a microelectromechanical system (MEMS) acoustic and/or pressure sensor. However, in some embodiments, the microphone 28 may comprise a traditional electret, coil, or condenser type microphone.

FIG. 2B shows an exemplary embodiment of a portable electronic device 30, which may comprise a smart phone, a smart watch, a laptop computer, a tablet computer, or the like. It will be appreciated the portable electronic device 30 may alternatively comprise stationary electronic devices such as the desktop computer. The portable electronic device 30 is configured to receive a phonocardiogram from the stethoscope 20 and classify it as being normal or abnormal using a machine learning model. In the illustrated embodiment, the portable electronic device 30 comprises a processor 32 operably connected with memory 34, transceivers 36, an I/O interface 38, and a display screen 39. It will be recognized by those of ordinary skill in the art that a “processor” includes any hardware system, hardware mechanism or hardware component that processes data, signals or other information. The processor 32 may include a system with a central processing unit, multiple processing units, dedicated circuitry for achieving functionality, or other systems.

The transceivers 36 at least includes a transceiver, such as a Bluetooth® transceiver, configured to communicate with the stethoscope 20, but may also include any of various other devices configured for communication with other electronic devices, including the ability to send communication signals and receive communication signals. In one embodiment, the transceivers 36 further include additional transceivers which are common to smart phones and/or smart watches, such as Wi-Fi transceivers and transceivers configured to communicate via for wireless telephony networks.

The I/O interface 38 includes software and hardware configured to facilitate communications with the one or more interfaces of the portable electronic device 30 including the display screen 39, as well as other interfaces such as tactile buttons, switches, and/or toggles, microphones, speakers, and connection ports. The display screen 39 may be an LED screen or any of various other screens appropriate for a portable electronic device. The I/O interface 38 is in communication with the display screen 39 and is configured to visually display graphics, text, and other data to the user via the display screen 39.

The memory 34 may be of any type of device capable of storing information accessible by the processor 32, such as a memory card, ROM, RAM, hard drives, discs, flash memory, or other computer-readable medium. The memory 34 is configured to store program instructions that, when executed by the processor 32, enable the portable electronic device 30 to perform various operations described elsewhere herein, including communicating with the stethoscope 20 to receive a phonocardiogram the heart of the person 12, processing the phonocardiogram to identify abnormal heart sounds, and informing the user in the event that abnormal heart sounds are detected. In at least one embodiment, the memory 34 is configured to store user data 40 that may include a user profile having demographic information such as name, age, gender, height, weight, and/or other information for the person 12. The user data 40 may further include medical history information, such as previously recorded phonocardiograms and information regarding any previously detected heart abnormalities.

The memory 34 is also configured to store program instructions corresponding to at least one machine learning model, in particular to a phonocardiogram classification model 42 and classification parameters 44 thereof. The processor 32 is configured to utilize the phonocardiogram classification model 42 to extract features from the phonocardiogram of the heart of the person 12 and to classify the phonocardiogram as being normal or abnormal. As used herein, the term “machine learning model” refers to a system or set of program instructions and/or data configured to implement an algorithm or mathematical model that predicts and provides a desired output based on a given input. It will be appreciated that parameters of a machine learning model are not explicitly programmed or the machine learning model is not necessarily designed to follow particular rules in order to provide the desired output for a given input. Instead, the machine learning model is provided with a corpus of training data from which identifies or “learns” patterns and statistical relationships or structures in the data, which are generalized to make predictions with respect to new data inputs. The classification parameters 44 include a plurality of values for parameters of the phonocardiogram classification model 42 which were learning during a training process.

The abnormal heart sound detecting system 10 with the phonocardiogram classification model 42 improves upon traditional cardiac auscultation methods. Particularly, the abnormal heart sound detecting system 10 is a non-invasive, cost-effective, and easy to use. This ease of use beneficial not only for use by physicians for in-office diagnoses, but also for use by non-expert individuals at home and for telemedicine applications. Thus, the system 10 can be of significant impact for early diagnosis of cardiac diseases, particularly for regions of the world that suffer from a shortage and geographic mal-distribution of skilled physicians. Additionally, the system 10 helps physicians make more confident decisions on heart abnormalities which can avoid the order of non-necessary tests and lead to great cost savings.

### Phonocardiogram Classification Model

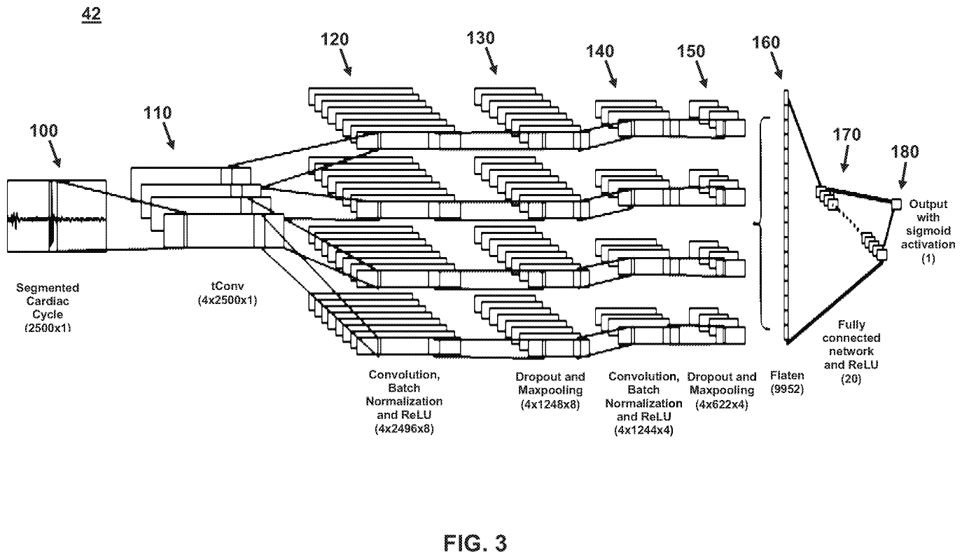

FIG. 3 shows an exemplary embodiment of the phonocardiogram classification model 42. The phonocardiogram classification model 42 is configured to receive a digital audio waveform corresponding segmented cardiac cycle 100. The phonocardiogram classification model 42 comprises at least one neural network, in particular at least one convolutional neural network (CNN), configured to extract features from a segmented cardiac cycle 100 and classify the segmented cardiac cycle 100 as being normal or abnormal.

The classification parameters 44 of the phonocardiogram classification model 42 comprise a plurality of kernel weights and/or filter values which are learned in a training process and used by the convolutional neural network(s) to extract features from the segmented cardiac cycle and to classify the segmented cardiac cycle 100 as being normal or abnormal. The phonocardiogram classification model 42 is trained using a dataset comprising a large number of phonocardiograms, each having a large number of cardiac cycles recorded therein. In at least one embodiment, each phonocardiogram of the dataset is labeled with a corresponding class label: normal or abnormal. One example of such a dataset is the 2016 PhysioNet/CinC Challenge dataset. In one embodiment, during training, Adam is used for stochastic optimization and binary cross-entropy is chosen as the loss function to be minimized. In at least one embodiment, the training process is performed on an external device, such as a server (not shown), and the resulting classification parameters 44 are provided to the portable electronic device 30 for storage in the memory 34 and usage thereat.

After the classification parameters 44 are learned in the training process, phonocardiogram classification model 42 can be used at the portable electronic device 30 to extract features from a segmented cardiac cycle 100 and classify the segmented cardiac cycle 100 as being normal or abnormal. Particularly, the portable electronic device 30 receives a digital audio waveform (hereinafter the “phonocardiogram”) from the stethoscope 20 corresponding to a phonocardiogram of the heart of the person 12. In one embodiment, in a pre-processing step, the processor 32 is configured to resample the phonocardiogram to a predetermined sample rate (e.g., 1000 Hz). In one embodiment, in a pre-processing step, the processor 32 is configured to apply a bandpass filter to eliminate extraneous frequencies that are unrelated to heart sounds (e.g., a band pass filter between 25 Hz and 500 Hz). The processor 32 of the portable electronic device 30 is configured to segment the phonocardiogram into one or more segmented cardiac cycles 100. In one embodiment, the processor 32 is configured to zero-pad the segmented cardiac cycles 100 to be a predetermined length (e.g., 2.5 seconds or 2500×1).

The phonocardiogram classification model 42 includes time-convolution (tConv) layers 110 configured to receive the segmented cardiac cycle 100 and decompose the segmented cardiac cycle 100 into a plurality of different time series corresponding to different frequency sub-bands. In the illustrated embodiment, the phonocardiogram classification model 42 includes four time-convolution layers 110, each configured to decompose the segmented cardiac cycle 100 into a time series corresponding to a respective frequency sub-band. Each decomposed time series has dimensions equal to that of the segmented cardiac cycle 100 (e.g., 2.5 seconds or 2500×1). Each time-convolution layer 110 is implemented as a one-dimensional convolutional neural network (1D-CNN) having a kernel which is learned during the training process.

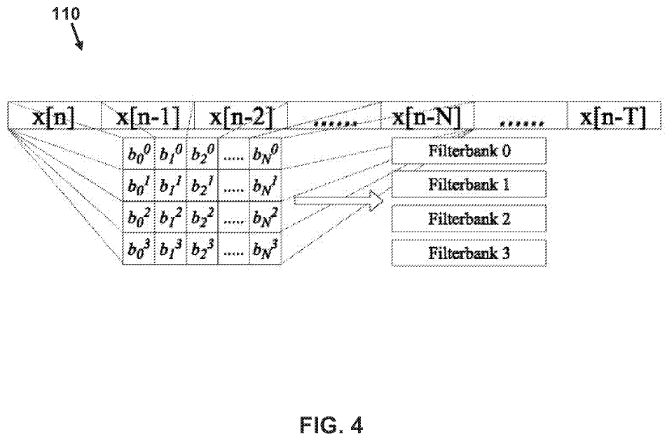

As illustrated in FIG. 4 and described below, the time-convolution layers 110 function as a Finite Impulse Response (FIR) filter-bank front-end, which learns the frequency characteristics of the FIR filters. Particularly, for a causal discrete-time FIR filter of order N with filter coefficients b₀, b₁, . . . , b_{N}, the output samples y[n] are obtained by a weighted sum of the most recent samples of the input signal x[n]. This can be expressed as:

$$\begin{matrix}y [ n ] = {b}_{0} x [ n ] + {b}_{1} x [ n - 1 ] + … + {b}_{N} x [ n - N ] \\ = {\sum }_{i = 0}^{N} {b}_{i} x [ n - i ] .\end{matrix}$$

Through a local connectivity pattern of neurons between adjacent layers, the 1D-CNN of the time-convolution layer 110 is configured to perform cross-correlation between its input x[n] (i.e., the segmented cardiac cycle 100) and its kernel. The output of the convolutional layer, with a kernel of odd length N+1, can be expressed as:

$$\begin{matrix}y [ n ] = {b}_{0} x [ n + \frac{N}{2} ] + {b}_{1} x [ n + \frac{N}{2} - 1 ] + … + {b}_{\frac{N}{2}} x [ n ] + … + \\ {b}_{N - 1} x [ n - \frac{N}{2} + 1 ] + {b}_{N} x [ n - \frac{N}{2} ] \\ = {\sum }_{i = 0}^{N} {b}_{i} x [ n + \frac{N}{2} - i ]\end{matrix}$$

where b₀, b₁, . . . , b_{N} are the kernel weights, x[n] is the input signal, y[n] are the output samples, and N is the order of the filter.

Considering a causal system, the output of the convolutional layer becomes:

$$y [ n - \frac{N}{2} ] = σ ( β + {\sum }_{i = 0}^{N} {b}_{i} x [ n - i ] )$$

where σ(.) is the activation function and β is the bias term. Therefore, a 1D convolutional layer with linear activation (i.e., σ(x)=x) and zero bias (i.e., β=0), acts as an FIR filter with an added delay of N/2.

In the embodiments of FIGS. 3 and 4, the time-convolution layers 110 include a plurality of different convolution layers, each having different learned filter characteristics and/or a different learned kernel weights. Particularly, as shown, the time-convolution layers 110 comprise four different time-convolution layers corresponding to the four frequency sub-bands of the segmented cardiac cycle 100. As shown in FIG. 4, a first time-convolution layer has an set of kernel weights b₀⁰, b₁⁰, b₂⁰ . . . , b_{N}⁰ and functions as a first filter bank. A second time-convolution layer has an set of kernel weights b₀¹, b₁¹, b₂¹ . . . , b_{N}¹ and functions as a second filter bank. A third time-convolution layer has an set of kernel weights b₀², b₁², b₂² . . . , b_{N}² and functions as a third filter bank. A fourth time-convolution layer has an set of kernel weights b₀³, b₁³, b₂³ . . . , b_{N}³ and functions as a fourth filter bank.

This FIR intuition of the time-convolution layers 110 discussed above provides new insights into the frequency and phase response of the kernel. Especially, large kernels can introduce significant phase distortion into their activations. The phase response of a filter indicates the phase shift in radians that each input component sinusoid will undergo. A convolutional kernel with non-linear phase response would introduce a temporal shift between the high frequency (e.g., murmurs) and low frequency (e.g., systole and diastole) patterns in the phonocardiogram signal.

To mitigate the effect, in at least one embodiment, one or more of time-convolution layers 110 have symmetric kernel weights around its center, such that the time-convolution layers 110 have a linear phase response. Particularly, in at least one embodiment, one or all of time-convolution layers 110 are trained with the constraint that their respective kernel weights are symmetric around their centers (i.e., b₀=b_{N}, b₁=b_{N-1}, b₂=b_{N-2}, etc.). This embodiment is referred to herein as a linear phase time-convolution layer (LP-tConv). We note that linear phase is the condition when the phase response of a filter is a linear function of frequency (excluding phase wraps at +/−π radians). A time-convolution layers 110 having a kernel with symmetric weights around its center have a linear phase response because they introduces an equal delay for all of the passing frequencies/patterns, ensuring no phase distortion.

In at least one further embodiment, one or more of the time-convolution layers 110 are configured to incorporate a forward-reverse convolution such that they have a zero phase response. This embodiment is referred to herein as a zero phase time-convolution layer (ZP-tConv). We note that a zero phase filter is a special case of a linear phase FIR filter in which the phase response is nullified.

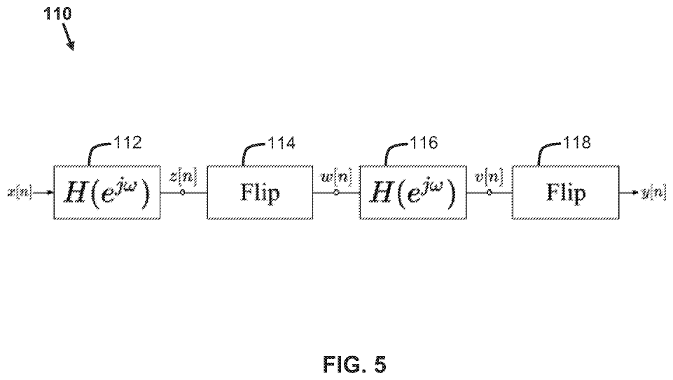

FIG. 5 shows on embodiment of a time-convolution layer 110 in which a forward-reverse convolution is incorporated, such that the time-convolution layer 110 has zero phase response. Particularly, a first time-convolution 112 is performed on the input signal x[n] to arrive at an first intermediate output z[n]. Next, a flip operation 114 is performed on the first intermediate output z[n] to arrive at a second intermediate output w[n]. Next, a second time-convolution 116 is performed on the second intermediate output w[n] to arrive at a third intermediate output v[n]. Finally, a flip operation 118 is performed on the third intermediate output v[n] to arrive at the final output y[n]. The operation of the ZP-tConv is summarized in the frequency domain as follows:

$$\begin{matrix}\begin{matrix}Y ( {e}^{j ω} ) = X ( {e}^{j ω} ) \cdot {H}^{*} ( {e}^{j ω} ) \cdot H ( {e}^{j ω} ) \\ = X ( {e}^{j ω} ) {\vert {e}^{j ω} \vert}^{2}\end{matrix} & \\ H ( {e}^{j ω} ) = \vert H ( {e}^{j ω} ) \vert ∠ H ( {e}^{j ω} ) & \end{matrix}$$

where X(e^{jω}) is the Fourier transform of the input signal x[n], Y(e^{jω}) is the Fourier transform of the final output y[n], and H(e^{jω}) is the Fourier transform of the impulse response of the kernel h[n]. We note that, the flip operation in time domain is equivalent to taking the complex conjugate in the frequency domain. Therefore, the effect of the ZP-tConv is a multiplication by the squared magnitude in the frequency domain.

The parameters of the time-convolution layers 110, in particular the kernel weights b₀, b₁, . . . , b_{N} are learned during the training process. In at least one embodiment, the kernel weights b₀, b₁, . . . , b_{N} are learned and/or updated with Stochastic Gradient Descent (SGD). In at least one embodiment, the kernel weights b₀, b₁, . . . , b_{N} are initialized based on equivalent FIR filter coefficients corresponding to band pass filters for a predetermined set of frequency sub-bands (e.g., 25-45, 45-80, 80-200, 200-500 Hz). In further embodiments, the kernel weights b₀, b₁, . . . , b_{N} are initialized randomly or with zero values.

It will be appreciated that the time-convolution layers 110 offer an improvement over traditionally implemented FIR filter-bank front-end because, rather than arbitrarily selecting cutoff frequencies for each frequency sub-band (e.g., 25-45, 45-80, 80-200, 200-500 Hz), the filter characteristics of each time-convolution layer 110 are learned based on training data. In this way, the time-convolution layers 110 decompose the segmented cardiac cycle 100 into more pathologically significant frequency sub-bands, thereby making the phonocardiogram classification model 42 more effective in distinguishing pathologic heart sounds.

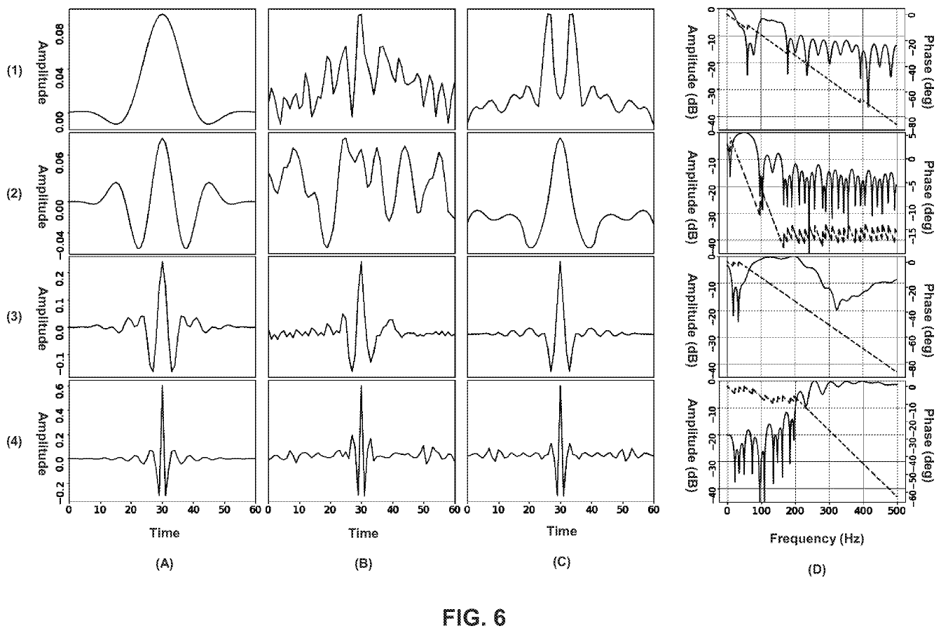

FIG. 6 shows decompositions of a segmented cardiac cycle 100 with an exemplary FIR filter bank, an exemplary tConv layer, and an exemplary LP-tConv layer, as well as magnitude and phase responses of the exemplary LP-tConv layer. Particularly, the plots in rows (1), (2), (3), and (4) correspond to the four respective input branches of time-convolution layers 110. The plots in column (A) correspond the decomposed frequency sub-bands using a FIR filter bank designed to implement band pass filters for the frequency sub-bands 25-45 Hz (1), 45-80 Hz (2), 80-200 Hz (3), and 200-500 Hz (4). The plots in column (B) correspond to the decomposed frequency sub-bands using a tConv layer having a learned kernel initialized based on the equivalent FIR coefficients for the sub-bands of column (A). The plots in column (C) correspond to the decomposed frequency sub-bands using a LP-tConv layer having a learned kernel initialized based on the equivalent FIR coefficients for the sub-bands of column (A). Finally, the plots in column (D) correspond to magnitude response (solid line) and phase response (dashed line) of the learned LP-tConv layer of column (C). As can be observed, the learned kernel for the higher frequency sub-bands are less affected by the training process after initialization, compared the lower frequency sub-bands.

Returning to FIG. 3, the time series output by the time-convolution layers 110, which correspond to the decomposed frequency sub-bands, are each fed into a respective input branch of the phonocardiogram classification model 42. In the illustrated embodiment, the phonocardiogram classification model 42 includes four branches. Each branch of the phonocardiogram classification model 42 includes a first convolutional layer 120, a first maxpooling layer 130, a second convolutional layer 140, and a second maxpooling layer 150. The phonocardiogram classification model 42 further includes a flattening layer 160, and a multilayer perceptron (MLP) network having hidden fully connected layer 170 and an output layer 180.

The first convolutional layer 120 is implemented as a convolutional neural network having a predetermined number of filters with a predetermined length and/or kernel size. The first convolutional layer 120 is configured to extract features of the respective frequency sub-band segment. In the illustrated embodiment, the first convolutional layer 120 of each branch has 8 filters of length and/or kernel size 5. The first convolutional layer 120 is followed by a Rectified Linear Unit (ReLU) activation of the output. In at least one embodiment, the first convolutional layer 120 is also followed by batch normalization and/or L2 regularization. After activation, the first maxpooling layer 130 pools and/or reduces the dimensionality of the output with a predetermined pool size (e.g., 2). In at least one embodiment, after the maxpooling, a dropout layer is applied to dropout a random set (e.g., 50%) of activations.

The second convolutional layer 140 is similarly implemented as a convolutional neural network having a predetermined number of filters with a predetermined length and/or kernel size. The second convolutional layer 140 is configured to extract features of the respective frequency sub-band segment. In at least one embodiment, the second convolutional layer 140 has fewer filters than the first convolutional layer 120. In the illustrated embodiment, the second convolutional layer 140 of each branch has 4 filters of length and/or kernel size 5. The second convolutional layer 140 is followed by a Rectified Linear Unit (ReLU) activation of the output. In at least one embodiment, the second convolutional layer 140 is also followed by batch normalization and/or L2 regularization. After activation, the second maxpooling layer 150 pools and/or reduces the dimensionality of the output with a predetermined pool size (e.g., 2). In at least one embodiment, after the maxpooling, a dropout layer is applied to dropout a random set (e.g., 50%) of activations.

The flattening layer 160 flattens and concatenates the outputs of each branch of the phonocardiogram classification model 42. After flattening and concatenation, the output is fed to the hidden fully connected layer 170 of the multilayer perceptron network. The hidden fully connected layer 170 is followed by a Rectified Linear Unit (ReLU) activation. In one embodiment, a dropout layer is applied to dropout a random set (e.g., 50%) of activations at the hidden fully connected layer 170. In one embodiment, L2 regularization is applied at the hidden fully connected layer 170. Finally, the output layer 180 of the multilayer perceptron network comprises a single neuron as output with sigmoid activation.

The phonocardiogram classification model 42 is configured to provide a prediction with respect to the segmented cardiac cycled 100 at the output layer 180. In the illustrated embodiment, the output layer 180 includes only a single neuron and thus provides a single output value. In particular, after sigmoid activation the output layer provides a probability (e.g., a value between 0 and 1) that the segmented cardiac cycled 100 is abnormal and/or probability that the segmented cardiac cycled 100 is normal. However, it will be appreciated that, in some embodiments, the output layer 180 may be configured to provide more than one output. For example, the output layer 180 may be configured to provide probabilities of various specific heart sound abnormalities, if the training data was classified and labeled with specific types of heart sound abnormalities.

As discussed above, a phonocardiogram generally comprises several cardiac cycles. Accordingly, the phonocardiogram classification model 42 may provide a predicted probability that the segmented cardiac cycle 100 is abnormal/normal for each segmented cardiac cycle 100. In one embodiment, the predicted probabilities of all the segmented cardiac cycles 100 are averaged to determine a final prediction with respect to the phonocardiogram.

In one embodiment, hyper-parameters of the phonocardiogram classification model 42 are tuned for optimal performance using a Tree of Parzen Estimators. Such hyper-parameters may include learning rate (e.g., 0.0012843784), learning rate decay (e.g., 0.00011132885), dropout after convolution layers (e.g., 50%), L2 regularization in convolution layers (e.g., 0.0486), and pool size (e.g., 2).

### Methods for Detecting Abnormal Heart Sounds

Methods for operating the abnormal heart sound detecting system 10 are described below. In particular, methods of operating the stethoscope 20 and/or the portable electronic device 30 to detect abnormal hearts sounds of a heart of a person 12 are described. In the description of the methods, statements that a method is performing some task or function refers to a controller or general purpose processor executing programmed instructions stored in non-transitory computer readable storage media operatively connected to the controller or processor to manipulate data or to operate one or more components in the abnormal heart sound detecting system 10 to perform the task or function. Particularly, the processors 22 of the stethoscope 20 and/or the processor 32 of the portable electronic device 30 above may be such a controller or processor. Alternatively, the controller or processor may be implemented with more than one processor and associated circuitry and components, each of which is configured to form one or more tasks or functions described herein. It will be appreciated that some or all of the operations the method can also be performed by a remote server or cloud processing infrastructure. Additionally, the steps of the methods may be performed in any feasible chronological order, regardless of the order shown in the figures or the order in which the steps are described.

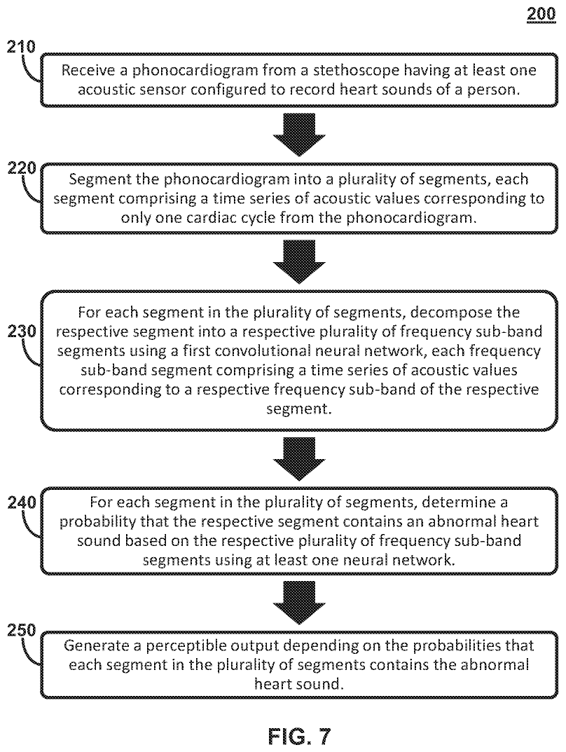

FIG. 7 shows a logical flow diagram for a method 200 operating an abnormal heart sound detecting system 10 to detect abnormal hearts sounds of a heart of a person 12. The method 200 improves upon the functioning of the abnormal heart sound detecting system 10 and, more particularly, the functioning of the processor 22 of the stethoscope 20 and/or the processor 32 of the portable electronic device 30, by advantageously utilizing the time-convolution layers 110 configured to decompose a segmented cardiac cycle of a phonocardiogram into pathologically significant frequency sub-bands. Additionally, the method 200 is a non-invasive, cost-effective, and easy to use. This ease of use beneficial not only for use by physicians for in-office diagnoses, but also for use by non-expert individuals at home and for telemedicine applications. Thus, the method 200 can be of significant impact for early diagnosis of cardiac diseases, particularly for regions of the world that suffer from a shortage and geographic mal-distribution of skilled physicians. Additionally, the method 200 helps physicians make more confident decisions on heart abnormalities which can avoid the order of non-necessary tests and lead to great cost savings.

The method 200 begins with a step of receiving a phonocardiogram from a stethoscope having at least one acoustic sensor configured to record heart sounds of a person (block 210). Particularly, with respect to the embodiments described in detail herein, the processor 22 of the stethoscope 20 is configured to operate the microphone 28 to record a phonocardiogram of the heart of the person 12, while the stethoscope 20, or at least the microphone 28 thereof, is placed near or against the chest of the person 12. The phonocardiogram is digitized and comprises a time series of acoustic values corresponding to heart sounds of the person 12. The processor 22 is configured to operate the transceiver 26 to transmit the recorded phonocardiogram to the portable electronic device 30. The processor 32 of the portable electronic device 30 is configured to operate the transceiver 36 to receive the phonocardiogram.

The method 200 continues with a step of segmenting the phonocardiogram into a plurality of segments, each segment comprising a time series of acoustic values corresponding to only one cardiac cycle from the phonocardiogram (block 220). Particularly, with respect to the embodiments described in detail herein, the processor 32 of the portable electronic device 30 is configured to segment the phonocardiogram into a plurality of segment cardiac cycles 100. In at least one embodiment, each segmented cardiac cycle 100 comprises a time series of acoustic values corresponding to only one cardiac cycle from the phonocardiogram. In one embodiment, the processor 32 is configured to zero-pad the segmented cardiac cycles 100 to be a predetermined length (e.g., 2.5 seconds). In one embodiment, in a pre-processing step, the processor 32 is configured to resample the phonocardiogram to a predetermined sample rate (e.g., 1000 Hz). In one embodiment, in a pre-processing step, the processor 32 is configured to apply a bandpass filter to eliminate extraneous frequencies that are unrelated to heart sounds (e.g., a band pass filter between 25 Hz and 500 Hz).

The method 200 continues with a step of, for each segment in the plurality of segments, decomposing the respective segment into a respective plurality of frequency sub-band segments using a first convolutional neural network, each frequency sub-band segment comprising a time series of acoustic values corresponding to a respective frequency sub-band of the respective segment (block 230). Particularly, with respect to the embodiments described in detail herein, the processor 32 of the portable electronic device 30 is configured to decompose the segmented cardiac cycle(s) 100 into a plurality of frequency sub-band segments (e.g., four different frequency sub-band segments) using the time-convolution layers 110. Each frequency sub-band segment comprises a time series of acoustic values corresponding to a respective frequency sub-band of the segmented cardiac cycle(s) 100. Each frequency sub-band segment series has dimensions equal to that of the segmented cardiac cycle 100 (e.g., 2500×1).

As discussed above, each time-convolution layer 110 has a unique set of kernel weights b₀^{j}, b₁^{j}, b₂^{j} . . . , b_{N}^{j}, where j corresponds to the respective time-convolution layer 110. In at least one embodiment, the processor 32 is configured to determine each respective frequency sub-band segment based on a respective segmented cardiac cycle 100 using a different respective time-convolution layer 110. More particularly, in one embodiment, the processor 32 is configured to determine each respective frequency sub-band segment by calculating a cross-correlation between the respective segmented cardiac cycle 100 and the unique set of kernel weights b₀^{j}, b₁^{j}, b₂^{j} . . . , b_{N}^{j} of the different respective time-convolution layer 110. As a result, each time-convolution layer 110 generates a different pathologically significant frequency sub-band segment, due to unique filtering characteristics of each time-convolution layer 110. As discussed above, exemplary frequency sub-band segment comprises are shown in columns (B) and (C) of the FIG. 6.

In at least one embodiment, the time-convolution layers 110 are configured as linear phase time-convolution layers (LP-tConv). Particularly, to achieve a linear phase response, the ordered sequence of kernel weights b₀^{j}, b₁^{j}, b₂^{j} . . . , b_{N}^{j} of each time-convolution layer 110 have values that are symmetric about a center of the ordered sequence. In other words, the time-convolution layers 110 are trained with the constraints that b₀=b_{N}, b₁=b_{N-1}, b₂=b_{N-2}, etc. By providing symmetric kernel weights, the time-convolution layer 110 provides a linear phase response.

In at least one embodiment, the time-convolution layers 110 are configured as zero phase time-convolution layers (ZP-tConv), which incorporate forward-reverse convolution. Particularly, in one embodiment, the processor 32 is configured to determine each respective frequency sub-band segment by performing forward and reverse passes through the respective time-convolution layer 110. More particularly, the processor 32 is configured to determine a first intermediate output by calculating a cross-correlation between the respective segmented cardiac cycle 100 and the kernel weights b₀^{j}, b₁^{j}, b₂^{j} . . . , b_{N}^{j} of the respective time-convolution layer 110. Next, the processor 32 is configured to determine a second intermediate output by flipping the first intermediate output. Next, the processor 32 is configured to determine a third intermediate output by calculating a cross-correlation between the second intermediate output and the kernel weights b₀^{j}, b₁^{j}, b₂^{j} . . . , b_{N}^{j} of the respective time-convolution layer 110. Finally, the processor 32 is configured to determine the respective frequency sub-band segment by flipping the third intermediate output. By performing forward and reverse passes through the respective time-convolution layer 110, the time-convolution layer 110 provides a zero phase response.

With continued reference to FIG. 7, the method 200 continues with a step of, for each segment in the plurality of segments, determining a probability that the respective segment contains an abnormal heart sound based on the respective plurality of frequency sub-band segments using at least one neural network (block 240). Particularly, with respect to the embodiments described in detail herein, the processor 32 of the portable electronic device 30 is configured to determine a probability or probabilities that the segmented cardiac cycle(s) 100 contains an abnormal heart sound based on the frequency sub-band segments decomposed from the respective the segmented cardiac cycle 100 using at least one neural network. In the particular embodiments described herein, the processor 32 is configured to use the phonocardiogram classification model 42, which includes the first convolutional layer 120, the first maxpooling layer 130, the second convolutional layer 140, the second maxpooling layer 150, the flattening layer 160, and the multilayer perceptron (MLP) network having the hidden fully connected layer 170 and the output layer 180. However, it will be appreciated that in alternative embodiments, other types of machine learning models may be used to process the frequency sub-band segments to detect an abnormal heart sound in the segmented cardiac cycle(s) 100.

In at least one embodiment, the processor 32 is configured to provide each frequency sub-band segment to a respective branch of the phonocardiogram classification model 42. As discussed above, each branch includes the first and second convolutional layer 120 and 140, which are configured to extract features of the respective frequency sub-band segment. After activation and pooling, the processor 32 is configured to determine an intermediate output by flattening and concatenating the outputs of the branches using the flattening layer 160. Finally, at the output layer 180, the processor 32 is configured to determine the probability that the segmented cardiac cycle(s) 100 contains an abnormal heart sound based on the intermediate output using the multilayer perceptron network of the phonocardiogram classification model 42.

As discussed above, the phonocardiogram classification model 42 is configured to provide a prediction with respect to the segmented cardiac cycled 100 at the output layer 180 and a phonocardiogram generally comprises several cardiac cycles. In at least one embodiment, the processor 32 is configured to determine respective probabilities for each of a plurality of segmented cardiac cycle(s) 100. In one embodiment, the processor 32 is configured to determine an average of the determined probabilities.

Finally, the method 200 continues with a step of generating a perceptible output depending on the probabilities that each segment in the plurality of segments contains the abnormal heart sound (block 250). Particularly, with respect to the embodiments described in detail herein, the processor 32 of the portable electronic device 30 is configured to operate an output device to generate a perceptible output depending on the determined probability or probabilities that the segmented cardiac cycle(s) 100 contains an abnormal heart sound. In one embodiment, the processor 32 is configured to operate the output device to generate a perceptible output in response to the probability and/or the average of the probabilities exceeding a predetermined threshold. In at least one embodiment, the output device is the display screen 39 of the portable electronic device 30 and the perceptible output is a notification displayed on the display screen 39 which indicates that the phonocardiogram likely includes an abnormal heart sound. However, in other embodiments, the output device may be a speaker or light that is operated to generate a perceptible output indicating that the phonocardiogram likely includes an abnormal heart sound.

While the disclosure has been illustrated and described in detail in the drawings and foregoing description, the same should be considered as illustrative and not restrictive in character. It is understood that only the preferred embodiments have been presented and that all changes, modifications and further applications that come within the spirit of the disclosure are desired to be protected.

What is claimed is:

1. A method of detecting abnormal heart sounds in a phonocardiogram of a person, the method comprising:
receiving, with a processor, a first segment of the phonocardiogram, the first segment comprising a time series of acoustic values from the phonocardiogram;
decomposing, with the processor, the first segment into a plurality of frequency sub-band segments using a first convolutional neural network having a plurality of kernel weights that were learned in a training process of the first convolutional neural network, each frequency sub-band segment comprising a time series of acoustic values corresponding to a respective frequency sub-band of first segment;
determining, with the processor, a probability that the first segment contains an abnormal heart sound based on the plurality of frequency sub-band segments using at least one neural network; and
generating, with an output device, a perceptible output depending on the probability that first segment contains the abnormal heart sound.

2. The method according to claim 1, wherein the first segment of the phonocardiogram includes only one cardiac cycle of the phonocardiogram.

3. The method according to claim 2, the receiving the first segment of the phonocardiogram further comprising:
receiving, with a transceiver, the phonocardiogram from a stethoscope having at least one acoustic sensor configured to record heart sounds of the person; and
segmenting, with the processor, the phonocardiogram into a plurality of segments, each segment comprising a time series of acoustic values corresponding to only one cardiac cycle from the phonocardiogram, the plurality of segments including the first segment.

4. The method according to claim 3, further comprising, for each segment in the plurality of segments:
decomposing, with the processor, the respective segment into a respective plurality of frequency sub-band segments using the first convolutional neural network, each frequency sub-band segment comprising a time series of acoustic values corresponding to a respective frequency sub-band of the respective segment; and
determining, with the processor, a probability that the respective segment contains the abnormal heart sound based on the respective plurality of frequency sub-band segments using the at least one neural network.

5. The method according to claim 4 further comprising:
generating, with the output device, the perceptible output in response to an average of the probabilities that each segment in the plurality of segments contains the abnormal heart sound exceeding a predetermined threshold.

6. The method according to claim 1, wherein the plurality of frequency sub-band segments include at least four different frequency sub-band segments.

7. The method according to claim 1, wherein the first convolution neural network has a plurality of convolutional layers, the decomposing the first segment into the plurality of frequency sub-band segments further comprising:
determining each frequency sub-band segment in the plurality of frequency sub-band segments based on the first segment using a different respective convolutional layer in the plurality of convolutional layers.

8. The method according to claim 7, wherein each convolutional layer in the plurality of convolutional layers has a respective set of kernel weights in the plurality of kernel weights, the determining each frequency sub-band segment in the plurality of frequency sub-band segments further comprising:
determining the respective frequency sub-band segment by performing a cross-correlation between the first segment and the set of kernel weights of the different respective convolutional layer in the plurality of convolutional layers.

9. The method according to claim 8, wherein the set of kernel weights of each convolutional layer in the plurality of convolutional layers comprises an ordered sequence of kernel weights having values that are symmetric about a center of the ordered sequence.

10. The method according to claim 8, the determining each frequency sub-band segment in the plurality of frequency sub-band segments further comprising:
determining a first intermediate output by performing a cross-correlation between the first segment and the set of kernel weights of the different respective convolutional layer in the plurality of convolutional layers;
determining a second intermediate output by flipping the first intermediate output;
determining a third intermediate output by performing a cross-correlation between the second intermediate output and the set of kernel weights of the different respective convolutional layer in the plurality of convolutional layers; and
determining the respective frequency sub-band segment by flipping the third intermediate output.

11. The method according to claim 1, wherein the at least one neural network is a second convolutional neural network having a plurality of input branches, the determining of the probability that the first segment contains the abnormal heart sound further comprising:
providing each frequency sub-band segment in the plurality of frequency sub-band segments to a respective input branch of the plurality of input branches, each input branch in the plurality of input branches having at least one convolutional layer configured to extract features of the respective frequency sub-band segment; and
determining the probability that the first segment contains the abnormal heart sound based on outputs of the plurality of input branches.

12. The method according to claim 11, wherein the second convolutional neural network has a multilayer perceptron network, the determining of the probability that the first segment contains the abnormal heart sound further comprising:
determining an intermediate output by flattening and concatenating the outputs of the plurality of input branches; and
determining the probability that the first segment contains the abnormal heart sound based on the intermediate output using the multilayer perceptron network.

13. The method according to claim 1, the generating the perceptible output further comprising:
displaying a notification on a display screen depending on the probability that first segment contains the abnormal heart sound.

14. A system for detecting abnormal heart sounds in a phonocardiogram of a person, the system comprising:
a stethoscope having at least one acoustic sensor configured to record the phonocardiogram of the person and a transceiver configured to transmit the phonocardiogram; and
a portable electronic device having a processor, an output device, and a transceiver, the processor being configured to:
operate the transceiver to receive the phonocardiogram from the stethoscope;
segment the phonocardiogram into a plurality of segments, each segment comprising a time series of acoustic values corresponding to only one cardiac cycle from the phonocardiogram;
for each segment in the plurality of segments:
decompose the respective segment into a respective plurality of frequency sub-band segments using the first convolutional neural network, each frequency sub-band segment comprising a time series of acoustic values corresponding to a respective frequency sub-band of the respective segment; and
determine a probability that the respective segment contains the abnormal heart sound based on the respective plurality of frequency sub-band segments using at least one neural network; and
operate the output device to generate a perceptible output depending on the probabilities that each segment in the plurality of segments contains the abnormal heart sound.

15. The system according to claim 14, wherein the first convolution neural network has a plurality of convolutional layers, the processor being configured to, for each segment in the plurality of segments:
determine each frequency sub-band segment in the plurality of respective frequency sub-band segments based on the respective segment using a different respective convolutional layer in the plurality of convolutional layers.

16. The system according to claim 15, wherein each convolutional layer in the plurality of convolutional layers has a respective set of kernel weights in the plurality of kernel weights, the processor being configured to, for each frequency sub-band segment in the plurality of respective frequency sub-band segments:
determining the respective frequency sub-band segment by performing a cross-correlation between the respective segment and the set of kernel weights of the different respective convolutional layer in the plurality of convolutional layers.

17. The system according to claim 16, wherein the set of kernel weights of each convolutional layer in the plurality of convolutional layers comprises an ordered sequence of kernel weights having values that are symmetric about a center of the ordered sequence.

18. The system according to claim 16, the processor being configured to, for each frequency sub-band segment in the plurality of respective frequency sub-band segments:
determine a first intermediate output by performing a cross-correlation between the first segment and the set of kernel weights of the different respective convolutional layer in the plurality of convolutional layers;
determine a second intermediate output by flipping the first intermediate output;
determine a third intermediate output by performing a cross-correlation between the second intermediate output and the set of kernel weights of the different respective convolutional layer in the plurality of convolutional layers; and
determine the respective frequency sub-band segment by flipping the third intermediate output.

19. The system according to claim 14, the processor being configured to:
operate the output device to generate the perceptible output in response to an average of the probabilities that each segment in the plurality of segments contains the abnormal heart sound exceeding a predetermined threshold.

20. The system according to claim 14, wherein:
the at least one neural network is a second convolutional neural network, the second convolutional neural network having a plurality of input branches, each input branch in the plurality of input branches having at least one convolutional layer, the second convolutional neural network having a multilayer perceptron network; and
the processor is configured to, for each segment in the plurality of segments:
provide each frequency sub-band segment in the plurality of frequency sub-band segments to a respective input branch of the plurality of input branches, the at least one convolutional layer of each input branch configured to extract features of the respective frequency sub-band segment;
determine an intermediate output by flattening and concatenating outputs of the plurality of input branches; and
determine the probability that the first segment contains the abnormal heart sound based on the intermediate output using the multilayer perceptron network.
